(19) United States
(12) Patent Application Publication
(10) Pub. No.: US 2020/0178999 A1
(43) Pub. Date: Jun. 11, 2020

(54) AN ULTRASONIC CUTTING DEVICE FOR OSTEOTOMY

(71) Applicant: MECTRON S.P.A., Carasco (GE) (IT)

(72) Inventors: Elisa STABILINI, Leivi (GE) (IT); Andrea CHIRICO, Sestri Levante (GE) (IT); Nicolo CERISOLA, Chiavari (GE) (IT); Giorgio BOZZINI, Genova (GE) (IT)

(73) Assignee: MECTRON S.P.A., Carasco (GE) (IT)

(21) Appl. No.: 16/615,696

(22) PCT Filed: May 29, 2018
(86) PCT No.: PCT/IB2018/053791

§ 371 Date: Nov. 21, 2019

(30) Foreign Application Priority Data
May 31, 2017 (IT) 102017000059460

(51) Int. Cl. A61B 17/32

(52) U.S. Cl. CPC A61B 17/320068; A61B 2017/320084; A61B 2017/320075

(57) ABSTRACT

An ultrasonic cutting device for osteotomy is provided. The device has a blade extending in a longitudinal direction, an attachment member connected through an extension member with a handpiece configured to generate vibrations. The attachment member is connected to a proximal end of the blade which has a plurality of cutting elements formed on a distal portion thereof. The cutting elements have an arcuate portion arranged at a free end of the distal portion in the longitudinal direction and the arcuate portion has a continuous cutting edge with a plurality of triangular shaped teeth on opposite sides of the blade. The overall configuration of the distal portion of the blade is symmetrical relative to the longitudinal axis and progressively widens from the arcuate portion towards the proximal portion of the blade in the portion where the teeth are formed.

### TECHNICAL FIELD OF THE INVENTION

The present invention relates generally to the field of osteotomy cutting devices. More particularly, the invention relates to a minimally invasive blade cutting device for ultrasound osteotomy.

### BACKGROUND

In the field of surgery, it is often necessary to cut bone tissues, for example, for the installation of prostheses or for the application of screws, nails and, more generally, support elements for the repair of damaged bones following traumas of various nature. Cutting of bone tissues is also necessary during spinal and cranial surgery.

Among the cutting devices for osteotomy, ultrasound devices are extremely common, which include a vibration generator with an ultrasonic frequency, typically of the order of a few tens of kHz, for example 20 kHz or higher, to which an appropriately shaped blade can be connected through a special attachment and an extension.

Known ultrasonic cutting devices comprise a flat blade which develops in a longitudinal direction and is shaped like a hacksaw or a chisel. In this case, the vibration generator is configured to generate longitudinal vibrations, parallel to the blade development direction, which favour the cutting action with reciprocating movement exerted manually by a surgeon.

The use of ultrasonic cutting devices is preferred over that of traditional, manual or motorized cutting devices, because the ultrasonic vibrations favour the penetration of the blade reducing the force required by a surgeon, which allows greater precision in making a cut.

Examples of ultrasonic cutting devices are described in patents EP1311197, U.S. Pat. Nos. 5,261,922, 5,695,510, 8,343,178 and in the international patent publication WO2006/059120 A1.

Ultrasonic cutting devices can be improved as regards the blade guide and penetration features, both considering the increasingly felt need for a high cutting precision, and the fact that the blade must generally cut through bone tissues of varying consistency and thickness without damaging the underlying organs and/or tissues, especially in the case of spinal and cranial surgery, in which damage to the dura mater should be minimized.

The need to provide ultrasonic cutting devices which allow obtaining high cutting precisions independently of the manual skill of a surgeon is also strongly felt.

### SUMMARY OF THE INVENTION

The technical problem posed and solved by the present invention is therefore that of providing an improved ultrasonic cutting device which allows overcoming the drawbacks mentioned above with reference to the prior art.

This problem is solved by an ultrasonic cutting device according to claim 1.

Preferred features of the present invention are defined by the dependent claims.

The ultrasonic cutting device for osteotomy according to the invention comprises a blade and an attachment member configured to be connected to a longitudinal vibration generator. The blade comprises a proximal portion connected to the attachment member and a distal portion which carries a plurality of cutting elements. Such cutting elements comprise a portion of arcuate shape obtained at the free end of the blade in the longitudinal direction and on which there is a continuous cutting profile. The cutting elements further comprise a plurality of teeth obtained on both sides of the blade symmetrically with respect to a longitudinal axis thereof. The teeth are aligned with each other along incident directions with respect to the longitudinal axis of the blade, so that the overall configuration of the distal portion is that of a wedge which widens from the arcuate portion in the proximal direction.

The main advantage provided by the invention is that of combining a continuous cutting profile with a pair of discontinuous cutting profiles respectively consisting of a plurality of teeth, in which each cutting profile has a specific function. In particular, the continuous cutting profile obtained at the free end in the longitudinal direction serves for guiding the device during the initial penetration step in the bone tissue, and offers a surgeon a good tactile feedback due to the continuous contact between the latter and the blade. The teeth arranged in alignment along directions which are incident with respect to the longitudinal axis of the blade allow instead to progressively widen the slit produced initially in the bone by the arcuate portion, without requiring a surgeon to move the blade sideways.

In other words, with respect to known ultrasonic cutting devices, the one according to the invention allows a surgeon to concentrate more on the precision of the cut and its depth, being able to rely on a blade geometry which autonomously provides for widening the produced slit, as the cutting device is advanced into the bone tissue.

According to an embodiment of the invention, the cutting profile of the arcuate portion is formed as a chamfer in the thickness direction of the blade, which simplifies manufacture in particular considering the dimensions and millimetric thicknesses of the blades of the ultrasonic cutting devices for osteotomy.

According to an embodiment of the invention, the ultrasonic cutting device comprises a plurality of indentations formed in the longitudinal direction in the vicinity of the arcuate portion. The function of the indentations is to improve the effect of frontal penetration of the device.

Further advantages, features and operating methods of the present invention will be apparent from the following detailed description of some embodiments thereof, made by way of a non-limiting example.

### BRIEF DESCRIPTION OF THE FIGURES

Reference will be made to the figures of the accompanying drawings, in which:

FIGS. 1 and 2 schematically show a top plan view and a lateral view, respectively, of the osteotomy device according to the present invention;

FIG. 3 shows a longitudinal sectional view of the device in FIG. 1;

FIG. 4 shows a detail of FIG. 1;

FIG. 5 schematically shows a top plan view of the device for osteotomy according to a variant embodiment of the present invention;

FIG. 6 shows a detail of FIG. 5.

### DETAILED DESCRIPTION OF PREFERRED EMBODIMENTS OF THE INVENTION

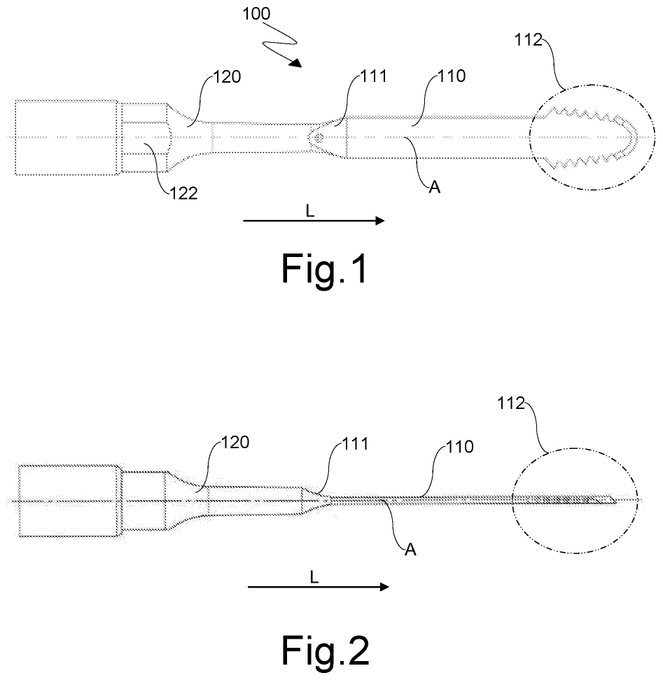

With reference initially to FIGS. 1 to 3, an ultrasonic cutting device according to the invention is generally indicated with reference numeral 100.

The device 100 comprises a blade 110 which extends in a longitudinal direction L from an attachment member or tang 120 of the device 100 itself configured for connection to a handpiece (not shown) provided with a generator of ultrasonic vibrations directed predominantly in the longitudinal direction L. The connection between the device 100 and the handpiece is not direct, but is carried out through a special rod-like extension (not shown) suitably protected from contact with the tissues by means of a sheath.

The blade 110 has a generally flat shape and the attachment member 120, of generally cylindrical shape, is connected to the blade 110 at a proximal portion 111 thereof, of thickness gradually decreasing in the longitudinal direction L.

The blade 110 and the attachment member 120 are made in one piece and made of stainless steel for medical use, for example X40CrMoVN16-2.

The blade 110 further comprises a distal portion provided with a plurality of cutting elements, as will be described in detail hereinafter. The distal portion is indicated as a whole with reference numeral 112.

The longitudinal sides or edges of the blade 110 are generally rectilinear and respectively parallel to a longitudinal axis A of the blade 110, which is in turn parallel to the longitudinal direction L.

The connection between the device 100 and the extension member can be of threaded type and for this purpose in the connection 120, for example, a threaded hole 121 is made, for example, in the longitudinal direction. To allow the manoeuvres for screwing and unscrewing the attachment member 120 with respect to the extension member, on its outer surface there are for example at least two diametrically opposed flat surfaces 122 for manoeuvring.

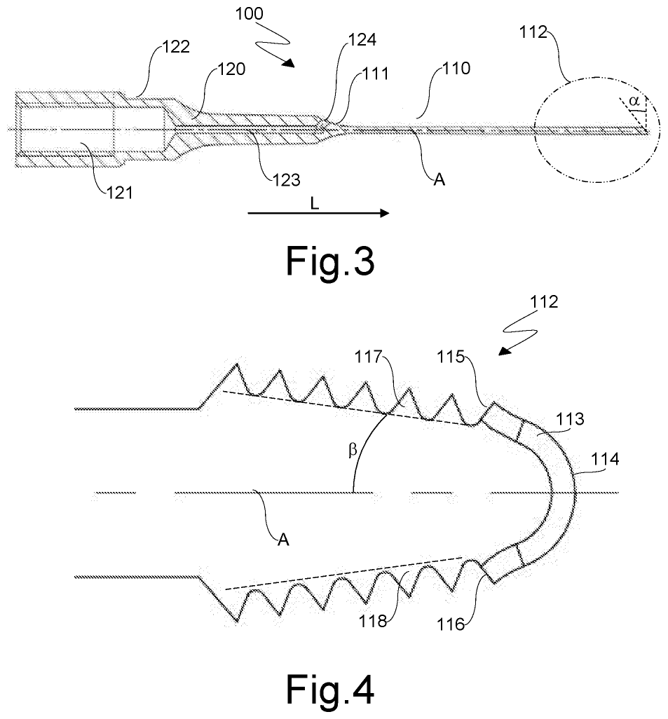

With particular reference to the longitudinal section in FIG. 3, the attachment member 120 further comprises a channel 123 which extends in the longitudinal direction L from the hole 121 towards the proximal portion 111 of the blade 110 and flows at an irrigation aperture 124 through which is possible to deliver a refrigerant fluid, for example a saline solution, provided by a remote supply source (not shown) connected to the handpiece.

In use, the blade 110 of the ultrasonic cutting device 100 is placed in the vicinity of the part to be cut without touching any surface. The vibration generator and the supply source of the refrigerant fluid are then started, and subsequently the blade 110 is brought into contact with the reference bone surface in the surgical site.

The blade 110 is moved alternately back and forth in the longitudinal direction L with respect to the bone tissue progressively penetrating therein. The refrigerant fluid delivered by the irrigation aperture 124 allows the removal of heat from the surgical site, favouring the penetration of the blade 110.

Referring now to FIG. 4, the distal portion 112 of the blade 110 comprises an arcuate portion 113 formed at the free end in the longitudinal direction L. The arcuate portion 113 develops symmetrically with respect to the longitudinal axis A of the blade 110, for example over an arc of about 180°.

The arcuate portion 113 comprises a continuous cutting profile 114 formed for example as a bevel in the thickness direction of the blade 110, as shown in the longitudinal section in FIG. 3. The inclination angle α of the chamfer is preferably comprised between 30° and 45°.

The continuous cutting profile 114 of the arcuate portion 113 allows obtaining a high blade guiding precision during the execution of a cut and offers a surgeon a good tactile feedback due to the continuous contact between the blade 110 and the bone tissue.

The arcuate portion 113 further comprises at its opposite ends two edges 115, 116 which join to the sides of first teeth 117, 118 of respective sets of teeth, all equal to each other, formed on both sides of the blade 110.

The teeth 117, 118 each have an isosceles triangular plan view and are preferably longitudinally spaced from each other by respective grooves which facilitate the discharge of the fragments of bone material during the cut.

The teeth 117, 118 formed on each side of the blade 110 are aligned with each other along incident directions with respect to the longitudinal axis A, schematically shown by broken lines. The inclination angle β of such incident directions is preferably comprised between 5° and 10°. The overall plan configuration of the distal portion 112 is symmetrical with respect to the longitudinal axis A and, in the section where the teeth 117, 118 are formed, progressively widens from the arcuate portion 113 towards the proximal portion 111 of the blade 110. The distal portion 112 terminates beyond the teeth 117, 118, where the blade 110 has a smaller width and a rectangular plan shape.

It will be understood that, during the execution of a cut, the conformation of the distal portion 112 causes a gradual widening of the slit created by the blade 110, which allows a surgeon to concentrate more on the cutting direction and its depth to the benefit of precision of execution of the same cuts.

Moreover, this particular configuration of the distal portion 112 contributes to the elimination of the bone material removed in synergy with a transversal component of the vibratory movement, or perpendicular to the longitudinal direction L and to the axis A of the ultrasonic device 100.

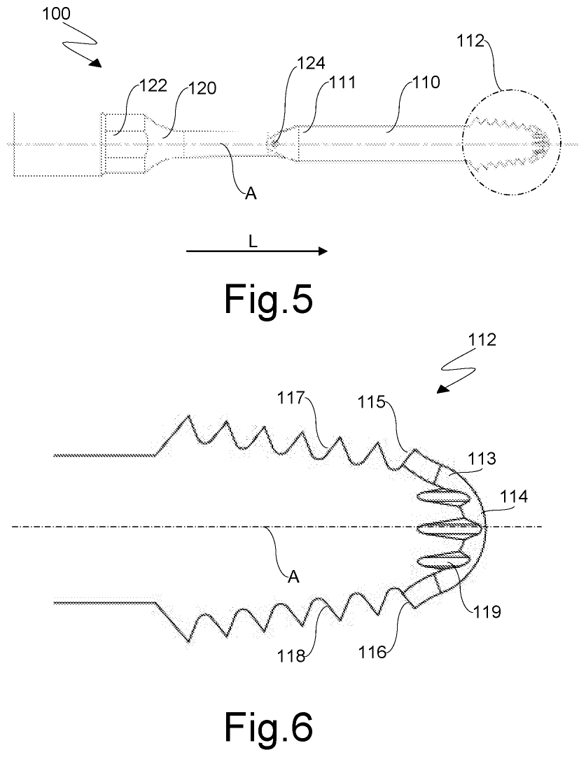

Referring now to FIGS. 5 and 6, according to a preferred embodiment of the invention, the ultrasonic cutting device 100 comprises a plurality of indentations 119 formed in the distal portion 112.

The indentations 119, for example three in the illustrated embodiment, are formed in the distal portion 112 of the blade 110 in the area surrounded by the arcuate portion 113 and extend parallel to the longitudinal axis A up to the chamfer 114.

The function of the indentations 119 is to facilitate the penetration of the device 100 into the bone tissue. More particularly, the portions of bone tissue located at the grooves of the indentations 119 are removed from the transverse component of the vibratory motion transmitted to the ultrasonic device 100, thus facilitating penetration into the bone tissue. In other words, the indentations 119 allow “removing” the bone material by freeing the space for the advancement of the device 100.

The present invention has been described thus far with reference to preferred embodiments thereof. It is understood that other embodiments may exist that relate to the same inventive scope, as defined by the scope of protection of the following claims.

1. An ultrasonic cutting device for osteotomy, said device comprising:
a blade extending in a longitudinal direction (L);
an attachment member configured to be connected, through a suitable extension member, with a handpiece configured to generate vibrations that are mainly directed in said longitudinal direction (L), said attachment member being connected to a proximal end of said blade;
wherein the blade further comprises a plurality of cutting elements formed on a distal portion thereof,
wherein said cutting elements comprise:
an arcuate portion arranged at a free end of said distal portion in the longitudinal direction (L), said arcuate portion having a continuous cutting edge;
a plurality of teeth having a triangular shape, said teeth being formed on opposite sides of the blade symmetrically with respect to a longitudinal axis (A) thereof, the teeth being aligned with each other along respective directions that are incident relative to said longitudinal axis (A),
the overall configuration of the distal portion of the blade being symmetrical relative to the longitudinal axis (A) and progressively widening from the arcuate portion towards the proximal portion of the blade in the portion where the teeth are formed.

2. The ultrasonic cutting device according to claim 1, wherein the arcuate portion extends over an arc about 180° symmetrically with respect to the longitudinal axis (A).

3. The ultrasonic cutting device according to claim 1, wherein the continuous cutting edge of the arcuate portion is a chamfer formed in the direction of the blade thickness.

4. The ultrasonic cutting device according to claim 3, wherein an inclination angle (α) of said chamfer is comprised between 30° and 45°.

5. The ultrasonic cutting device according to claim 1, further comprising a plurality of indentations formed in the distal portion of the blade surrounded by the arcuate portion, said indentations extending parallel to the longitudinal axis (A) up to the chamfer.

6. The ultrasonic cutting device according to claim 1, wherein, according to a top plan view, each tooth has the shape of an isosceles triangle.

7. The ultrasonic cutting device according to claim 6, wherein the teeth are spaced longitudinally from each other by respective grooves.

8. The ultrasonic cutting device according to claim 1, wherein the directions along which the teeth are aligned are inclined relative to the longitudinal axis (A) by an inclination angle (β) of between 5° and 10°.

9. The ultrasonic cutting device according to claim 1, wherein the attachment member comprises a channel for the feeding of a refrigerant fluid and an irrigation aperture for dispensing said fluid, said irrigation aperture being formed close to the proximal portion of the blade.
